(19) United States
(12) Patent Application Publication
(10) Pub. No.: US 2012/0271106 A1
(43) Pub. Date: Oct. 25, 2012

(54) SELF-PROPELLED DEVICE FOR ENDOSCOPE

(75) Inventors: Shinichi YAMAKAWA, Kanagawa (JP); Tsuyoshi ASHIDA, Kanagawa (JP); Takayuki NAKAMURA, Kanagawa (JP); Yasunori OHTA, Kanagawa (JP); Charles Alan BRANTINGHAM, St. Paul, MN (US)

(73) Assignee: FUJIFILM CORPORATION, Tokyo (JP)

(21) Appl. No.: 13/454,769

(22) Filed: Apr. 24, 2012

Related U.S. Application Data
(60) Provisional application No. 61/478,846, filed on Apr. 25, 2011.

(51) Int. Cl. A61B 1/00

(52) U.S. Cl. 600/114

(57) ABSTRACT

A self-propelled device nips an endless belt between a worm wheel and a driven roller. The endless belt is circulated by rotating the worm wheel, and thereby an insertion section of an endoscope advances and retreats inside a body cavity. The driven roller has a width larger than the worm wheel, and is provided with a pair of flanges at both ends. The worm wheel is disposed between the pair of flanges. When the endless belt is nipped between the worm wheel and the driven roller, the endless belt is deformed by the pair of flanges.

### BACKGROUND OF THE INVENTION

1. Field of the Invention

The present invention relates to a self-propelled device for an endoscope that assists in insertion of the endoscope into a body cavity.

2. Description of the Related Art

Endoscopes are widely used for observation or medical treatment in body cavities. This kind of endoscope is provided with an insertion section to be inserted into a body cavity, and an operation section for operating the insertion section. When the operation section is operated, the insertion section is inserted into the body cavity. In the endoscope, the insertion section is inserted into the body cavity while operating the operating section to curve a tip portion of the insertion section, however such insertion procedure requires extensive experience. For example, when the insertion section is inserted into a part like a sigmoid colon or a transverse colon which is not fixed to the inside of the body cavity, the insertion procedure requires a level of skill. If the skill is not enough, it results in considerable suffering to a patient. In view of this, a self-propelled device for an endoscope that propels the endoscope in an insertion direction inside the intestinal tract is proposed as disclosed in Japanese Patent Translation Publication No. 2009-513250. In this apparatus, a hollow toroidal rotary body is attached to a tip of an insertion section of an endoscope, and the rotary body is circulated in a longitudinal direction of the insertion section, whereby the insertion section is pulled to the depths of the intestinal tract. The rotary body is in contact with a driving roller that is provided between an outer periphery of the insertion section and the rotary body, and circulates in accordance with the rotation of the driving roller.

However, in the apparatus described in Japanese Patent Translation Publication No. 2009-513250, frictional force between the driving roller and the rotary body is not sufficient, and therefore there is a problem in that the driving roller runs idle.

### SUMMARY OF THE INVENTION

An object of the present invention is to provide a self-propelled device for an endoscope that can enhance frictional force between a driving roller and a rotary body, and thereby preventing the driving roller from running idle.

In order to achieve the above object, a self-propelled device for an endoscope of the present invention includes an attachment portion, a rotary body, a driving roller, a driven roller, and a flange. The attachment portion is detachably mounted on an insertion section of an endoscope. The rotary body is formed into a hollow toroidal shape or obtained by forming a belt in a ring shape. The driving roller is disposed to come in contact with the rotary body, and circulates and moves the rotary body. The driven roller is disposed to face the driving roller across the rotary body, and the rotary body is nipped between the driven roller and the driving roller. The driven roller rotates in accordance with the circulation of the rotary body. The flange is formed on at least one of the driven roller and the driving roller. In order to deform a part of the rotary body being nipped between the driven roller and the driving roller, the flange satisfies the following equation: R1+R2+D>L. Here, R1 is radius of the flange of the driving roller with the flange, or radius of the roller of the driving roller without flange. R2 is radius of the flange of the driven roller with the flange, or radius of the roller of the driven roller without flange. D is thickness of the rotary body being nipped. L is distance between shafts of the driving roller and the driven roller.

It is preferable that a first flange and a second flange are formed at both ends of the driven roller. The driving roller has a width smaller than the driven roller, and is disposed between the first and second flanges. The rotary body is deformed by the driven roller and the driving roller.

Preferably, a gear barrel, a worm gear, and a worm wheel are further included. The gear barrel rotates about a central axis of the insertion section. The worm gear is formed on an outer periphery of the gear barrel. The worm wheel as the driving roller is meshed with the worm gear and rotates about an axis perpendicular to the central axis of the insertion section.

The worm wheel preferably has a tooth row formed on its outer periphery with a tooth tip of the tooth row being tilted with respect to an axis of rotation of the worm wheel. The frictional force between the rotary body and the driving roller is increased by suppressing the rotary body toward the flange by a thrust load generated due to the tilt of the tooth tip.

Preferably, a linear projection is formed on a surface of the rotary body that is in contact with the driven roller. The projection is formed to pass through a center in a width direction of an outer periphery of the driven roller in accordance with the circulation of the rotary body. In order to penetrate the projection into, a groove is preferably formed at the center in the width direction of the outer periphery of the driven roller. A height of the projection may be larger than a depth of the groove. The height of the projection may be equal to or smaller than the depth of the groove.

It is also possible that a linear projection is formed on a surface of the rotary body that is in contact with the driving roller. The projection is formed to pass through a center in a width direction of an outer periphery of the driving roller in accordance with the circulation of the rotary body. The groove is formed at the center in the width direction of the outer periphery of the driving roller so that the projection penetrates into the groove.

A protrusion maybe formed at a center in a width direction of an outer periphery of the driven roller so as to press the projection toward the groove.

Preferably, the attachment portion has an opening through which the insertion section is inserted. The attachment portion is mounted on an outer periphery of the insertion section as the insertion section is inserted through the opening.

The rotary body may be an endless belt that is obtained by forming a belt in a ring shape. A plurality of the rotary bodies is disposed at regular intervals about a central axis of the insertion section.

It is preferable that a first flange and a second flange are formed at both ends of the driving roller. The driven roller has a width smaller than the driving roller, and is disposed between the first and second flanges. The rotary body is deformed by the driving roller with the flanges and the driven roller, thereby increasing the frictional force between the rotary body and the driving roller.

According to the present invention, the flange is provided on at least one of the driven roller and the driving roller, and the rotary body is nipped between the driven roller and the driving roller in a state where it is being deformed. Owing to this, the frictional force between the rotary body and the driving roller is enhanced, whereby the idle running of the driving roller is prevented.

### BRIEF DESCRIPTION OF THE DRAWINGS

The above object and advantages can be easily understood by those skilled in the art by reading the detailed description of the preferred embodiments of the invention with reference to the attached drawings:

FIG. 1 is a schematic view of an endoscope system;

FIG. 2 is a perspective view of an insertion section of the endoscope and a main body of a self-propelled device;

FIG. 3 is an exploded view of the main body of the self-propelled device;

FIG. 4 is an exploded view of an attachment portion;

FIG. 5 is a cross-sectional view of the main body sectioned at a plane perpendicular to a central axis CL;

FIG. 6 is a cross-sectional view taken along VI-VI line in FIG. 5;

FIG. 7 is a cross-sectional taken along VII-VII line in FIG. 6;

FIG. 8 is an explanatory view illustrating an example in which a specification of a driven roller is changed;

FIG. 9 is an explanatory view illustrating an example in which specifications of a driving roller, the driven roller, and an endless belt are changed;

FIG. 10A is an explanatory view illustrating the relationship between a linear projection and a groove;

FIG. 10B is an explanatory view similar to FIG. 10A where the linear projection is made higher;

FIG. 11 is an explanatory view illustrating an example in which specifications of the driving roller and the driven roller are changed; and

FIG. 12 is another explanatory view illustrating an example in which specifications of the driving roller and the driven roller are changed.

### DESCRIPTION OF THE PREFERRED EMBODIMENTS

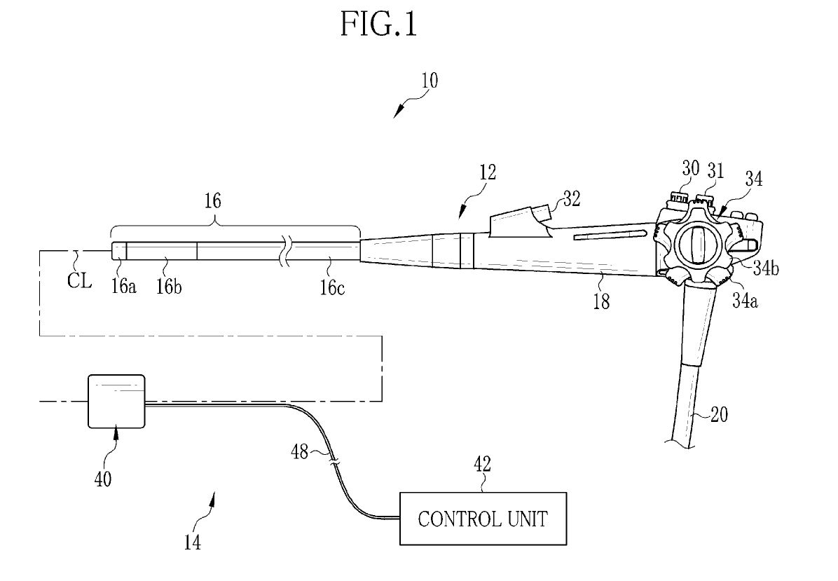

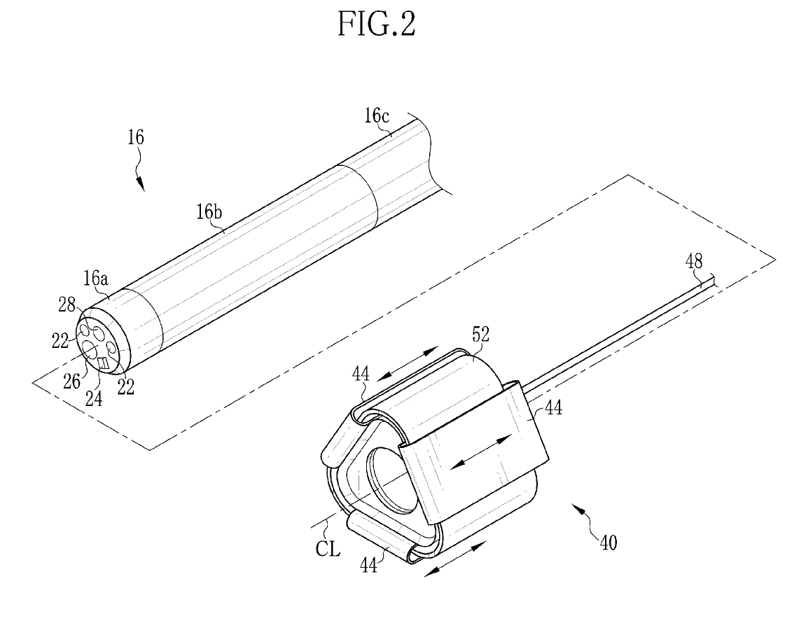

In FIGS. 1 and 2, an endoscope system 10 is constituted of an electronic endoscope 12 and a self-propelled device (endoscope insertion assisting device) 14 mounted on the electronic endoscope 12. The electronic endoscope 12 is constituted of an insertion section 16, an operation section 18, a universal cord 20, a processor (not shown), a light source unit (not shown), an air/water sending device (not shown), and the like. The insertion section 16 is inserted into a body cavity (e.g. large intestine). The operation section 16 is connected to a rear end of the insertion section 12. The operation section 16 is connected to the processor, the light source unit, and the air/water sending device via the universal cord 20.

The insertion section 16 is composed of a hard distal portion 16a, a curved portion 16b, and a flexible tube portion 16c provided in a sequence in this order from a distal (front) end. The distal portion 16a is provided with a pair of illumination window 22 for emitting illumination light from the light source unit to a site to be observed, an air/water nozzle 24 for injecting air or water supplied from the air/water sending device toward an observation window, and a forceps outlet 26 for exposing a tip of a medical instrument such as an electrosurgical knife which has inserted through a later-described forceps inlet 32.

The distal portion 16a is also provided with the observation window 28 for taking in an image of the site to be observed inside a body. Behind the observation window 28, an objective optical system and a solid-state imaging device such as a CCD type or CMOS type image sensor are provided. The solid-state imaging device is connected to the processor (not shown) by a signal cable that is inserted through the insertion section 16, the operation section 18, and the universal cord 20. The processor controls the drive of the solid-state imaging device to capture images of the site to be observed, and displays the obtained images on a monitor (not shown).

The curved portion 16b is capable of being curved, and is curved from right to left or up and down in accordance with the operation of the operation section 18. Owing to this, the distal portion 16a can be pointed to a desired direction. The flexible tube portion 16c is deformable by a helical coil or the like, and formed to have a length of several meters so that the distal portion 16a can reach a target site inside the body cavity.

The operation section 18 is provided with an air/water sending button 30, 31 for injecting air or water from the air/water nozzle 24 and the forceps inlet 32 through which the medical instrument is inserted. The operation section 18 is also provided with an angle knob 34. The angle knob 34 is constituted of two operation dials 34a, 34b layered one another. When the back dial 34a is rotated, the curved portion 16b is curved up and down through a wire, and when the front dial 34b is rotated, the curved portion 16b is curved right and left through the wire.

The self-propelled device 14 is mounted on the endoscope 12 and helps the insertion section 16 of the endoscope 12 advancing and retreating inside body canals. The self-propelled device 14 is equipped with a main body 40 and a control unit 42. The main body 40 is mounted on the tip of the insertion section 16 and inserted into the body cavity. The control unit 42 is disposed outside the body cavity and controls the drive of the main body 40.

The main body 40 is equipped with endless belts (rotary body) 44. The endless belt 44 is formed from, for example, biocompatible plastics, such as polyvinyl chloride, polyamide resin, fluororesin, and polyurethane, and has flexibility. Three endless belts 44 are arranged 120 degrees apart, e.g., evenly spaced around a central axis CL of the insertion section 16. The endless belts 44 are supported by a later-described support barrel 52 in a circulating manner, and circulate in a direction parallel to the central axis CL, that is, a longitudinal direction of the insertion section 16 indicated by an arrow in FIG. 2. Owing to the circulation of the endless belts 44, the insertion section 16 is provided with propulsive force.

A torque wire 48 for supplying a driving force to the endless belt 44 and a tube (not shown) that covers the torque wire 48 are provided at a rear end of the main body 40. Front ends of the torque wire 48 and the tube are connected to the main body 40, and rear ends of the same are connected to the control unit 42.

The control unit 42 is provided with a motor (not shown) for rotating the torque wire 48, and a control section (not shown) for adjusting the direction and speed of rotation of the motor. The rotation of the endless belt 44 can be restricted, that is, the direction and speed of propulsion of the insertion section 16 can be adjusted by controlling the control section.

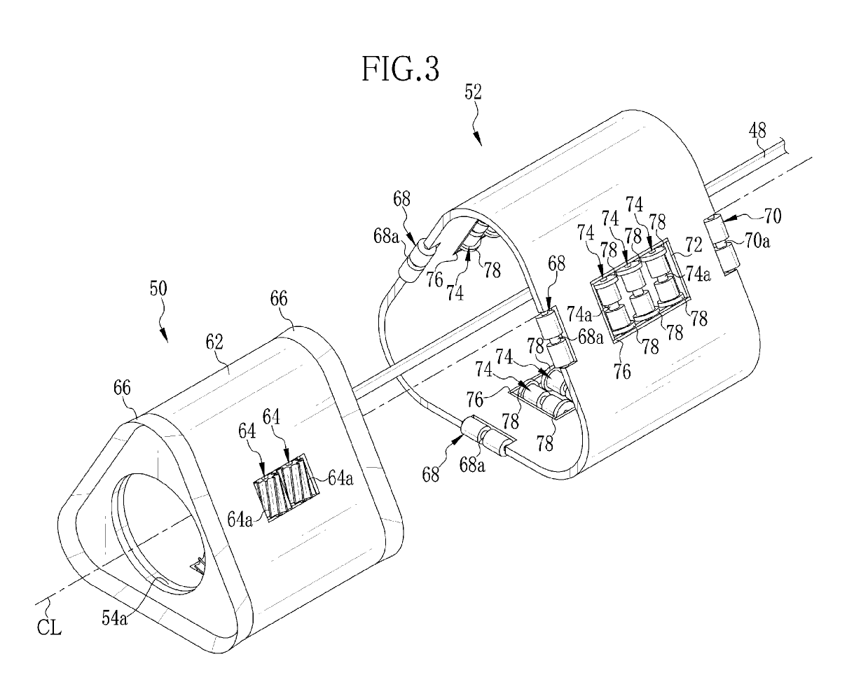

Hereinafter, the configuration of the main body 40 will be described in detail with reference to FIGS. 3 to 7. Note that illustration of the endless belts 44 is omitted in FIG. 3. As shown in FIG. 3, the main body 40 is equipped with an attachment portion 50 and a belt support barrel 52. The attachment portion 50 is detachably provided to the insertion section 16. The belt support barrel 52 is mounted on the outside of the attachment portion 50 and supports the endless belts 44.

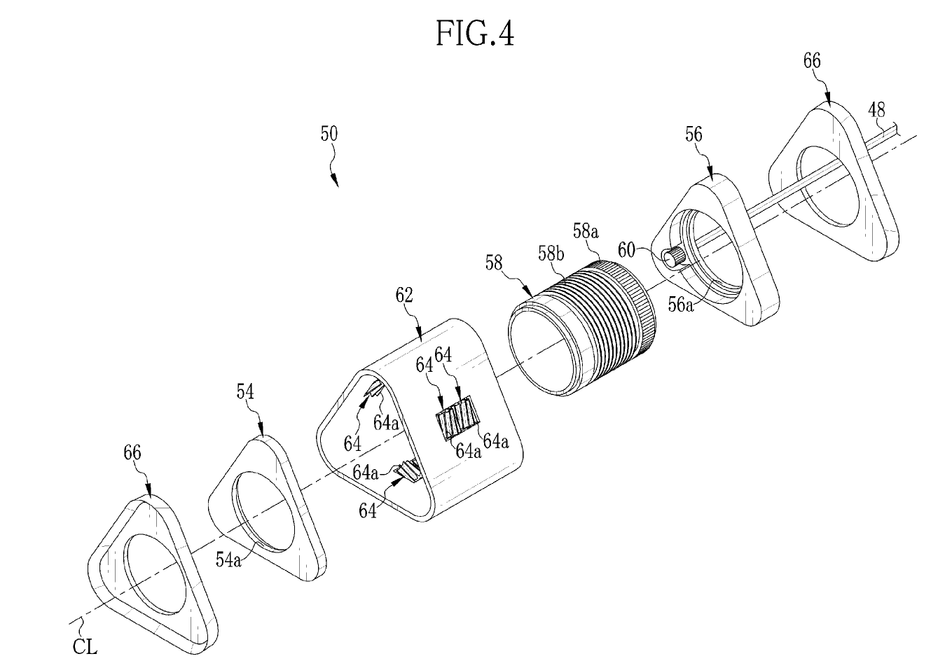

As shown in FIG. 4, the attachment portion 50 is equipped with a front plate 54 and a rear plate 56 respectively fixed on the inside of front and rear ends of a wheel support barrel 62. The front plate 54 and the rear plate 56 are formed with an opening 54a and an opening 56a, respectively through which the insertion section 16 is inserted. The attachment portion 50 is mounted on an outer periphery of the insertion section 16 so as not to fall out by fitting the insertion section 16 into the openings 54a and 56a.

A gear barrel (driving barrel) 58 is disposed between the front plate 54 and the rear plate 56 inside the wheel support barrel 62. The gear barrel 58 is formed in a cylindrical shape surrounding the insertion section 16. Between the front plate 54 and the rear plate 56, the gear barrel 58 is rotatably supported about the central axis CL. The rear plate 56 rotatably supports a pinion gear 60 that is attached to a front end of the torque wire 48. The pinion gear 60 meshes with a spur gear 58a formed on an outer periphery of a rear end of the worm gear 58, and the gear barrel 58 rotates in accordance with the rotation of the pinion gear 60.

The wheel support barrel 62 is formed in a substantially triangular tubular shape. An inner periphery of a front end of the wheel support barrel 62 is fitted to an outer periphery of the front plate 54, and an inner periphery of a rear end of the wheel support barrel 62 is fitted to an outer periphery of the rear plate 56, thereby integrating the wheel support barrel 62 with the front plate 54 and the rear plate 56. Three square through holes are formed through a flat side wall of the wheel support barrel 62 such that they are arranged 120 degrees apart around the central axis CL. A pair of two worm wheels: front and rear worm wheels 64 is disposed at each through hole.

Each of the worm wheel 64 is formed in a substantially cylindrical shape, and rotatably held about an axis perpendicular to the central axis CL. Tooth rows 64a are formed on an outer periphery of the worm wheel 64. A tooth tip of each tooth row 64a is tilted with respect to an axis of rotation of the worm wheel 64. The tooth rows 64a mesh with worms 58b formed on the outer periphery of the gear barrel 58. The worm wheel 64 rotates in accordance with the rotation of the gear barrel 58.

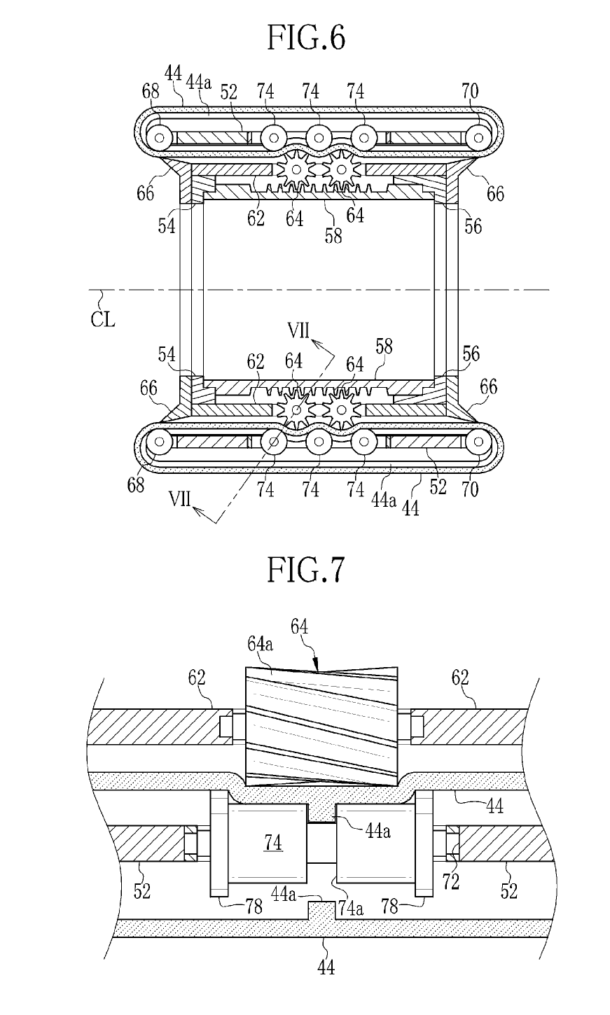

Front and rear ends of the attachment portion 50 are provided with wipers 66. The wipers 66 are provided to fill a gap between the attachment portion 50 and the endless belts 44, and an outer periphery of each wiper 66 is pressed against the endless belts 44 (see FIG. 6). When the endless belts 44 rotate, the wipers 66 slidingly contact with the endless belts 44, and thereby preventing foreign substances from entering (drawing-in) between the endless belts 44 and the attachment portion 50.

Referring back to FIG. 3, the belt support barrel 52 is formed in a triangular tubular shape having a larger diameter than the wheel support barrel 62. Three front rollers 68 are formed on a front end of the belt support barrel 52 such that they are arranged 120 degrees apart around the central axis CL. Rear rollers 70 corresponding to the respective front rollers 68 are formed on a rear end of the belt support barrel 52. The front rollers 68 and the rear rollers 70 are rotatably held about an axis perpendicular to the central axis CL. A groove portion 68a is formed at a center in a width direction of an outer periphery of each of the front rollers 68, and a groove portion 70a is formed at a center in a width direction of an outer periphery of each of the rear rollers 70.

Between the front rollers 68 and the rear rollers 70, through holes 72 are formed through a side wall of the belt support barrel 52. A set of three driven rollers: front, middle, and rear driven rollers 74 is disposed at each through hole 72. Each of the driven rollers 74 is rotatably held about an axis perpendicular to the central axis CL. In addition, a groove portion 74a is formed at a center in a width direction of an outer periphery of each of the driven rollers 74 (see FIG. 7). Note that the set of three driven rollers 74 is attached to a holder 76 first, and the holder 76 is attached to each of the through holes 72.

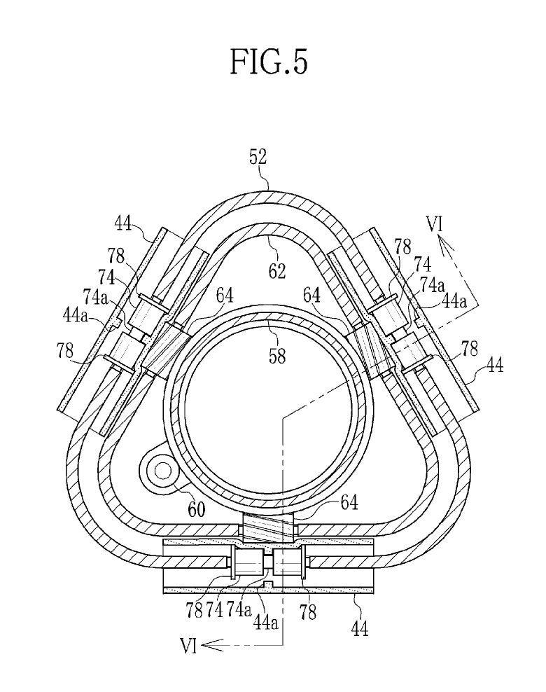

As shown in FIGS. 5 and 6, each endless belt 44 is bridged between the front roller 68 and the rear roller 70 such that the endless belt 44 covers a flat part of the side wall of the belt support barrel 52. A linear projection 44a is formed at a center in a width direction of an inner surface of the endless belt 44. The linear projection 44a is formed over the entire circumference of the endless belt 44, and fitted into the groove portion 68a of the front roller 68, the groove portion 74a of the driven roller 74, and the groove portion 70a of the rear roller 70. Owing to this, movement of the endless belt 44 in its width direction (movement about the central axis CL) is restricted.

In this way, the belt support barrel 52 holding three endless belts 44 is mounted on the outside of the attachment portion 50. When the belt support barrel 52 is mounted on the attachment portion 50, the driven rollers 74 and the worm wheels 64 are alternately arranged. Then, the endless belt 44 is nipped between the worm wheels 64 and the driven rollers 74 as well as being pressed against the worm wheels 64 by the driven rollers 74, and circulates and moves in accordance with the rotation of the worm wheels 64. Since each of the worm wheels 64 has relatively low teeth, the worm wheels 64 work as the driving rollers with ribs.

As described above, since the worm wheels 64 and the driven rollers 74 are alternately arranged (that is, the front worm wheel 64 is disposed between the front and middle driven rollers 74, and the rear worm wheel 64 is disposed between the middle and rear driven rollers 74), the back-and-forth motion of the belt support barrel 52 is restricted.

Moreover, as shown in FIG. 7, the driven roller 74 has a larger width as compared to the worm wheel 64, and has a pair of flanges 78 at both sides in its width direction. The width between the flanges 78 is larger than the width of the worm wheel 64, and the driven roller 74 is disposed such that the worm wheel 64 is kept between the flanges 78. Owing to this, movement of the driven roller 74 in its width direction (rotation of the belt support barrel 52 about the central axis CL) is restricted. In addition, when the endless belt 44 is nipped between the worm wheels 64 and the driven rollers 74, the endless belt 44 is deformed to correspond to the shape of a gap between the worm wheels 64 and the driven rollers 74.

Thus, in the self-propelled device 14, each of the driven rollers 74 is provided with the flanges, and the endless belt 44 is nipped between the worm wheels 64 and the driven rollers 74 with the endless belt 44 deformed. Owing to this, the endless belt 44 is strongly pressed against the worm wheel 64 by a periphery of the worm wheel 64 and the flanges 78 as a whole, which enhances frictional force of the endless belt 44, whereby the idle running of the worm wheel 64 is prevented.

Here, radius of roller of the worm wheel 64 is defined as R1, radius of the flanges 78 of the driven roller 74 is defined as R2, and thickness of the endless belt 44 being nipped is defined as D. If the endless belt 44 is compressible, the thickness D is a value in a state being compressed. Additionally, distance between shafts of the worm wheel 64 and the driven roller 74 is defined as L. In order to deform the endless belt 44 by the flanges 78, the following equation is satisfied: R1+R2+D>L. Note that, if the worm wheel 64 is formed with the flanges, the radius of the flanges is R1. On the contrary, if the driven roller 74 is not formed with the flanges, the radius of the roller of the driven roller 74 is R2.

Particularly, the tooth tip of the worm wheel 64 is tilted with respect to the axis of rotation of the worm wheel 64. Therefore, the endless belt 44 is suppressed toward one side in the width direction of the worm wheel 64 by a thrust load due to the tilt of the tooth tip. In the self-propelled device 14, since the endless belt 44 that is suppressed by the thrust load due to the tilt of the tooth tip is stopped by the flanges 78, the tension of the endless belt 44 between the flanges 78 and the worm wheel 64 is enhanced, thereby the endless belt 44 is securely nipped (the frictional force between the worm wheel 64 and the endless belt 44 is enhanced).

Note that it is only necessary in the present invention that at least one of the driven roller 74 and the worm wheel 64 is provided with the flanges to prevent the idle running of the worm wheel 64, and therefore detail configurations may be appropriately changed. For example, although the above embodiment is explained with an example of providing three endless belts 44, the number of the endless belts 44 may be two or less, or four or more.

In addition, although the above embodiment is explained with an example where the endless belt 44 is used as the rotary body, a rotary body of hollow toroidal shape may also be used as described in the above-mentioned patent document.

Moreover, although the above embodiment is explained with an example where the endless belt 44 as the rotary body is bridged between the rollers 68, 70 provided at the front and rear ends of the belt support barrel 52, fixed guide members may be used instead of the rollers 68, 70. The guide members are preferably made of a material having a low frictional resistance with respect to the endless belt 44.

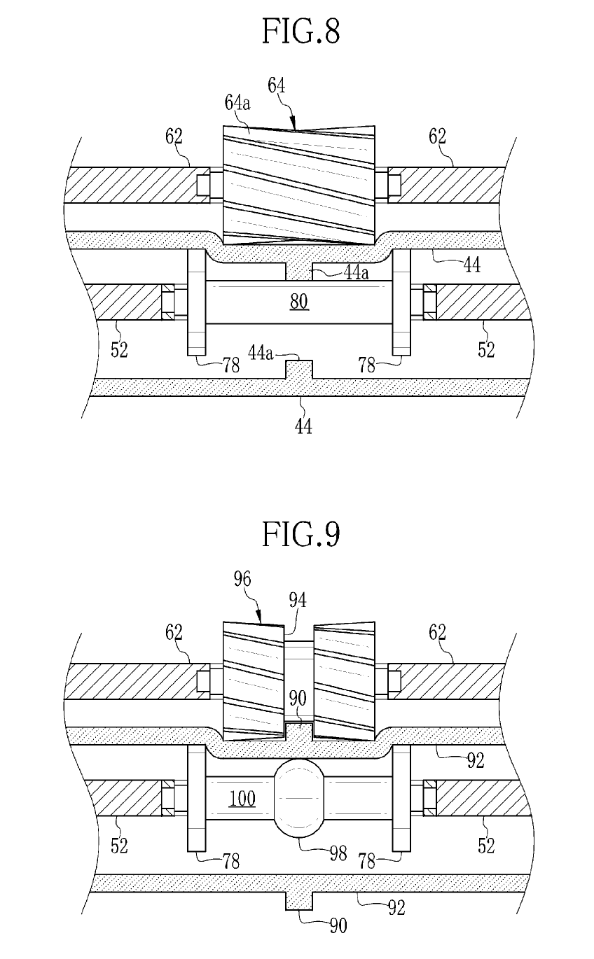

Additionally, although the above embodiment is explained with an example where the endless belt 44 is provided with the linear projection 44a to be fitted into the groove portion 74a of the driven roller 74, the linear projection 44a and the groove portion 74a can be eliminated. In an embodiment shown in FIG. 8, a flange roller with no groove as a driven roller 80 is used. The linear projection 44a is pressed by a shaft of the driven roller 80. Note that in embodiments shown in FIG. 8 and subsequent drawings, the same components as those of the above embodiment in terms of functions and structure are designated by the same reference numerals, and the description thereof is omitted.

It is also possible to use an endless belt 92 in combination with a worm wheel 96 and a driven roller 100, as shown in FIG. 9. The endless belt 92 is formed with a liner projection 90 on its outer surface. The worm wheel 96 has a groove portion 94 corresponding to the linear projection 90. The driven roller 100 has a protrusion 98 that presses the linear projection 90 against the groove portion 94. Note that, in FIG. 9, the protrusion 98 of the driven roller 100 may be eliminated. In this case, a diameter of the driven roller 100 is preferably made large so that a shaft of the driven roller 100 depresses the endless belt 92.

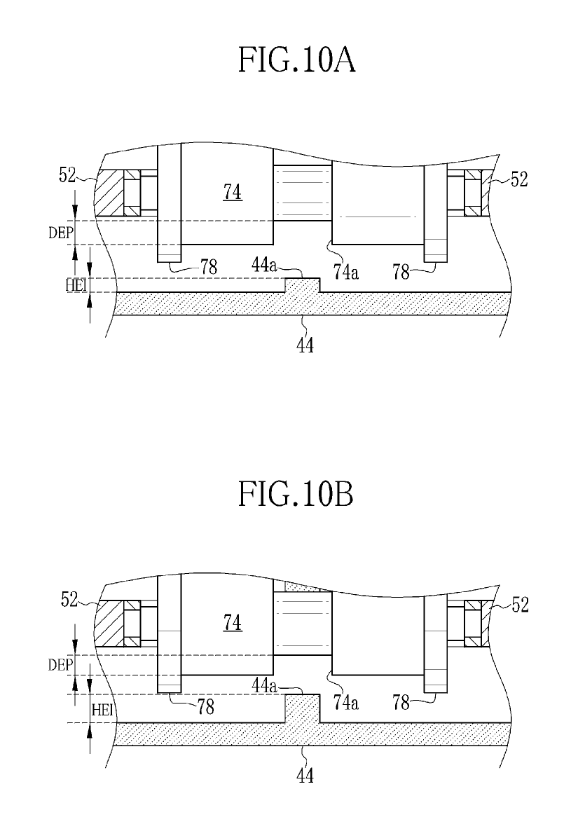

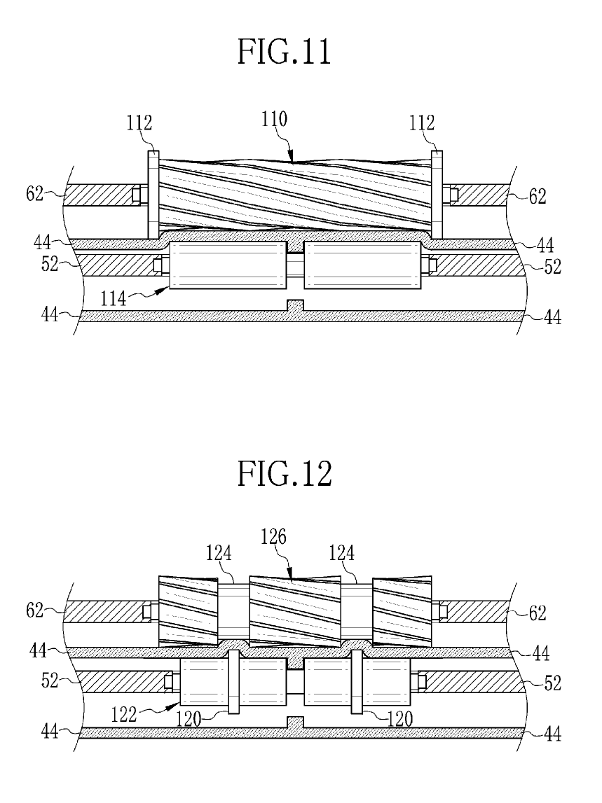

Moreover, specifications (height, width, and the like) of the linear projection and the groove portion are not limited to the above embodiments, and may be appropriately changed. For example, a height HEI of the linear projection 44a may be made smaller than a depth DEP of the groove portion 74a as shown in FIG. 10A, or the height HEI of the linear projection 44a may be made larger than the depth DEP of the groove portion 74a as shown in FIG. 10B. In the former case (FIG. 10A), since the linear projection 44a can freely move within the width of the groove portion 74a, the tension of the endless belt 44 between one of the flanges 78 and the worm wheel 64 is enhanced by the thrust load in accordance with the rotation of the worm wheel 64. Therefore, the endless belt 44 can be securely nipped.

On the other hand, in the latter case (FIG. 10B), the movement of the linear projection 44a within the width of the groove portion 74a is restricted. Owing to this, the effect of securely nipping the endless belt 44 by the thrust load is smaller than the case shown in FIG. 10A. Instead, the part of the endless belt 44 where the linear portion 44a is formed is more securely nipped, which prevents concentration of the load between the flanges 78 and the worm wheel 64. Therefore, it is possible to increase durability of the endless belt 44.

Note that, if the height HEI of the linear projection 44a is larger than the depth DEP of the groove portion 74a, the linear projection 44a needs to be deformable by compression. It is possible to elastically compress the linear projection 44a by pressing with the groove portion 74a, and thereby pressing the outer periphery of the driven roller 74 against the endless belt 44. For this configuration, since suppress strength by the groove portion 74a is also added, the suppress strength toward the endless belt 44 is enhanced, which increases the driving force of the worm wheel 64.

Additionally, although the driven roller 74 is provided with the flanges 78 in the above embodiments, it is also possible that a worm wheel 110 is provided with flanges 112, and used in combination with a driven roller 114 with no flanges. Of course, both the driven roller 114 and the worm wheel 110 can be provided with the flanges. When the driven roller 114 and the worm wheel 110 are both provided with the flanges, it is preferable that the flanges of both are alternately disposed in the width direction.

Moreover, the flanges are provided at both ends of the driven roller in the above embodiments, it is also possible to use a driven roller 122 having flanges formed inside from both ends, as shown in FIG. 12. In the example shown in FIG. 12, a worm wheel 126 having groove portions 124 formed at positions corresponding to the flanges 120 is used.

It is also possible to provide a fixed barrel inside the gear barrel 58, and fix this fixed barrel to the insertion section 16. In this case, the gear barrel 58 is rotatably supported by the fixed barrel. Moreover, the front plate 54, the rear plate 56, and the wiper 66 are fixed on the outer periphery of the fixed barrel.

In the above embodiments, the present invention is applied to an insertion assisting device of an electronic endoscope for medical diagnosis. However, the present invention maybe applied to insertion assisting devices of conduit observation instruments such as other endoscopes and ultrasonic probes for industrial use or the like.

Various changes and modifications are possible in the present invention and may be understood to be within the present invention.

What is claimed is:

1. A self-propelled device for an endoscope comprising:
an attachment portion detachably mounted on an insertion section of an endoscope;
a rotary body formed in a hollow toroidal shape or obtained by forming a belt in a ring shape;
a driving roller disposed to come in contact with said rotary body, said driving roller circulating and moving said rotary body;
a driven roller disposed to face said driving roller across said rotary body, said rotary body being nipped between said driven roller and said driving roller, said driven roller rotating in accordance with the circulation of said rotary body; and
a flange formed on at least one of said driven roller and said driving roller, said flange satisfying the following equation so as to deform a part of said rotary body being nipped between said driven roller and said driving roller:
R1+R2+D>L
wherein,
R1: radius of the flange of said driving roller with the flange, or radius of the roller of said driving roller without flange
R2: radius of the flange of said driven roller with the flange, or radius of the roller of said driven roller without flange
D: thickness of said rotary body being nipped
L: distance between shafts of said driving roller and said driven roller

2. The self-propelled device for an endoscope according to claim 1, wherein
said flange has a first flange and a second flange formed at both ends of said driven roller,
said driving roller has a width smaller than said driven roller, and is disposed between said first and second flanges; and
said rotary body is deformed by said driven roller and said driving roller.

3. The self-propelled device for an endoscope according to claim 1, further comprising:
a gear barrel rotating about a central axis of said insertion section;
a worm gear formed on an outer periphery of said gear barrel; and
a worm wheel being meshed with said worm gear and rotating about an axis perpendicular to said central axis of said insertion section, said worm wheel corresponding to said driving roller.

4. The self-propelled device for an endoscope according to claim 3, wherein
said worm wheel has a tooth row formed on its outer periphery with a tooth tip of said tooth row being tilted with respect to an axis of rotation of said worm wheel, and said frictional force between said rotary body and said driving roller is increased by suppressing said rotary body toward said flange by a thrust load generated due to the tilt of said tooth tip.

5. The self-propelled device for an endoscope according to claim 1, further comprising:
a linear projection formed on a surface of said rotary body that is in contact with said driven roller, said projection being formed to pass through a center in a width direction of an outer periphery of said driven roller in accordance with the circulation of said rotary body.

6. The self-propelled device for an endoscope according to claim 5, further comprising:
a groove formed at the center in the width direction of the outer periphery of said driven roller so that said projection penetrates into said groove.

7. The self-propelled device for an endoscope according to claim 6, wherein
a height of said projection is larger than a depth of said groove.

8. The self-propelled device for an endoscope according to claim 6, wherein
a height of said projection is equal to or smaller than a depth of said groove.

9. The self-propelled device for an endoscope according to claim 1, further comprising:
a linear projection formed on a surface of said rotary body that is in contact with said driving roller, said projection being formed to pass through a center in a width direction of an outer periphery of said driving roller in accordance with the circulation of said rotary body; and
a groove formed at the center in the width direction of the outer periphery of said driving roller so that said projection penetrates into said groove.

10. The self-propelled device for an endoscope according to claim 9, further comprising:
a protrusion formed at a center in a width direction of an outer periphery of said driven roller so as to press said projection toward said groove.

11. The self-propelled device for an endoscope according to claim 1, wherein
said attachment portion has an opening through which said insertion section is inserted, and is mounted on an outer periphery of said insertion section as said insertion section is inserted through said opening.

12. The self-propelled device for an endoscope according to claim 1, wherein
said rotary body is an endless belt that is obtained by forming a belt in a ring shape, and a plurality of said rotary bodies is disposed at regular intervals about a central axis of said insertion section.

13. The self-propelled device for an endoscope according to claim 1, wherein
said flange has a first flange and a second flange formed at both ends of said driving roller,
said driven roller has a width smaller than said driving roller, and is disposed between said first and second flanges; and
said rotary body is deformed by said driving roller and said driven roller.
